(19) United States
(12) Patent Application Publication
(10) Pub. No.: US 2009/0170148 A1
(43) Pub. Date: Jul. 2, 2009

(54) Nanog+, OCT-4+ Retinal Pigment Epithelial Stem Cells and Methods for Their Use and Manufacture

(76) Inventors: Yulia A. Smirnova, Moscow (RU); Rira D. Zinovieva, Moscow (RU); Lyubov Alexaudrovna Milyushina, Moscow (RU); Maria Anatolievna Alexandrova, Moscow (RU)

Correspondence Address: STEMEDICA CELL TECHNOLOGIES, INC, 5375 MIRA SORRENTO PLACE, SUITE 100, SAN DIEGO, CA, 92121, US

(21) Appl. No.: 12/189,589

(22) Filed: Aug. 11, 2008

Related U.S. Application Data
(60) Provisional application No. 60/955,598, filed on Aug. 13, 2007.

(51) Int. Cl. C12Q 1/02; C12N 5/00; C12N 5/06; C12N 5/08

(52) U.S. Cl. 435/29; 435/325; 435/348; 435/349; 435/350; 435/351; 435/366

(57) ABSTRACT

Retinal stem cells haying embryonic-like characteristics are disclosed. The retinal stem cells express one or more of the embryonic stem cell markers Nanog and OCT-4. The retinal stem cells may be obtained from retinal pigment epithelium. Methods of making and using retinal stem cells are also disclosed.

### PRIORITY

This application claims priority from provisional application Ser. No. 60/955,598 filed Aug. 13, 2007, the entire contents of which are incorporated by reference herein.

### FIELD OF THE INVENTION

The invention relates to the field of cell biology. More particularly the invention relates to stem cells and regenerative medicine. Still more particularly, the invention relates to a population of stem cells derived from retinal pigmented epithelium and methods for their use in research and therapeutic stem cell treatments.

### BACKGROUND

Retinal pigment epithelium (RPE) arises/from neuroectoderm and plays a key role in support of photoreceptor functions. The RPE serves a number of functions including: the uptake, processing, transport and release of vitamin A (retinol) and some of its visual cycle intermediates (retinoids); and the phagocytosis of rod and cone outer segment fragments that are shed from their distal ends. The RPE is unique in its apical and basolateral membrane polarity which is the reverse of most other epithelia (J Cell Sci Suppl. 1993; 17: 189-95). Several degenerative eye diseases, such as macular degeneration or retinitis pigmentosa, are associated with impaired RPE function that may lead to photoreceptor loss and blindness.

### SUMMARY OF THE INVENTION

The inventors have discovered a unique population of stem cells in the retina, particularly the retinal pigment epithelium. The discovered cells share a number of characteristics with embryonic stem cells including the expression of the pluripotent markers such as Nanog and OCT-4.

An object of the present invention is to provide a composition comprising purified retinal pigment epithelium stem cells, wherein said stem cells express at least one of OCT-4 and Nanog.

A further objective of the invention is to provide a clonal cell line of retinal pigment epithelium stem cells, wherein said stem cells express at least one of OCT-4 and Nanog.

A further objective of the invention is to provide a composition comprising pluripotent retinal pigment epithelium stem cells, wherein said stem cells express at least one of OCT-4 and Nanog.

A further object of the invention is to provide a method for testing the differentiation capacity of a test agent comprising providing composition comprising isolated pigmented retinal epithelial stem cells wherein said stem cells expresses at least one of Nanog and OCT-4, introducing to said composition said test agent, and determining the ability of said test agent to affect the differentiation of said stein cells

A further objective of the invention is to provide an assay for designing and/or screening for modulators or differentiation factors of cell differentiation, preferably neural cell differentiation and development.

A further object of the invention is to provide a pharmaceutical composition for use in regenerative stem cell therapy comprising retinal pigment epithelium stem cells in a pharmaceutically acceptable carrier, wherein said regenerative stern cell therapy is a treatment for a disease, disorder or abnormal state of the retina such as blindness, cytomegalovirus retinitis, uveitis, glaucoma, macular degeneration, retinitis pigmentosa, retinal degeneration, retinal detachment and cancers of the retina.

### BRIEF DESCRIPTION OF THE DRAWINGS

FIG. 1 is a drawing of the mammalian eye depicting various structures including the retinal pigment epithelium.

FIG. 2 is a table showing the expression pattern of transcription factors in eye structures in human fetal tissues at 5, 9 and 22 weeks.

FIG. 3 is a table showing a PCR analysis of transcription factors from a DNA library of cultured retinal cells from human fetus at 14 weeks.

FIG. 4a depicts a PCR analysis of transcription factors in adult human eye tissue, while FIG. 4b depicts a PCR analysis of transcription factors from a culture adult human retinal epithelium.

FIG. 5 depicts a staining analysis showing localized Nanog expression in human fetal eye at 12 weeks.

FIG. 6 depicts an anti-Nanog antibody stain of human fetal retina at 12 weeks.

FIG. 7 depicts an anti-Nanog antibody stain of human fetal retina at 22 weeks.

FIGS. 8a-c depict an in-situ antibody analysis of Nanog expression in retina from fetus at 12 weeks development.

FIGS. 9a-c depict an in-situ antibody analysis of OCT-4 expression in retina from fetus at 12 weeks development.

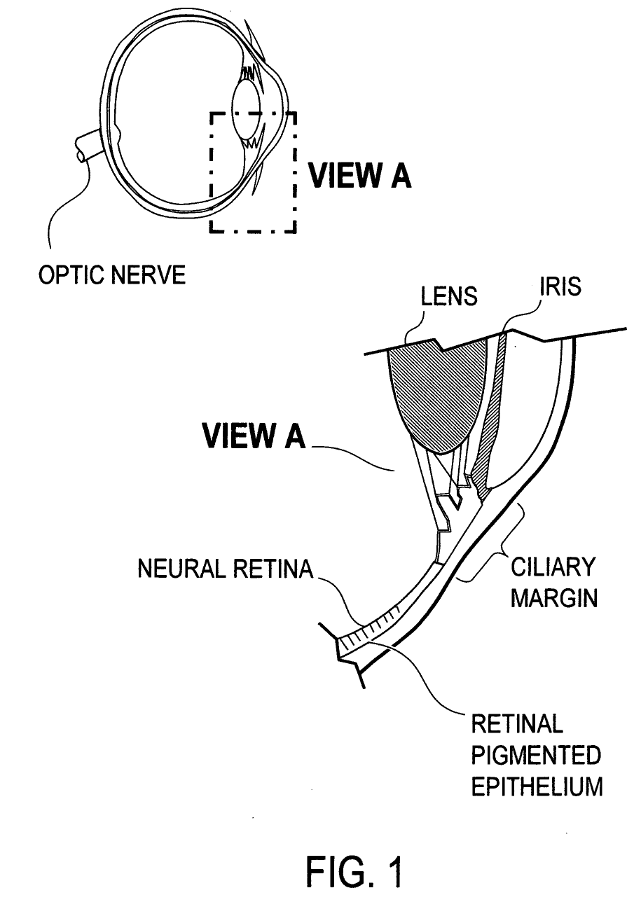

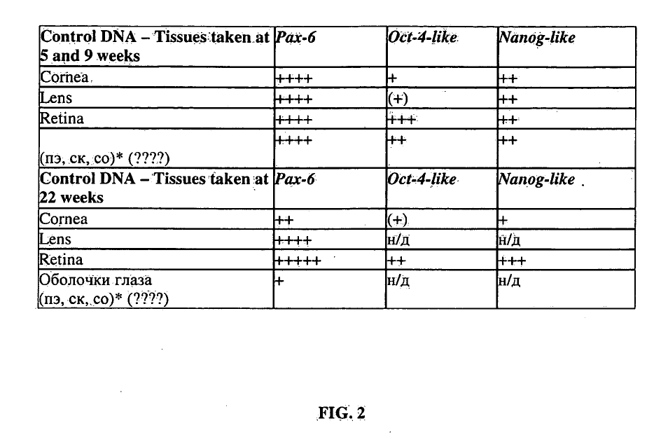

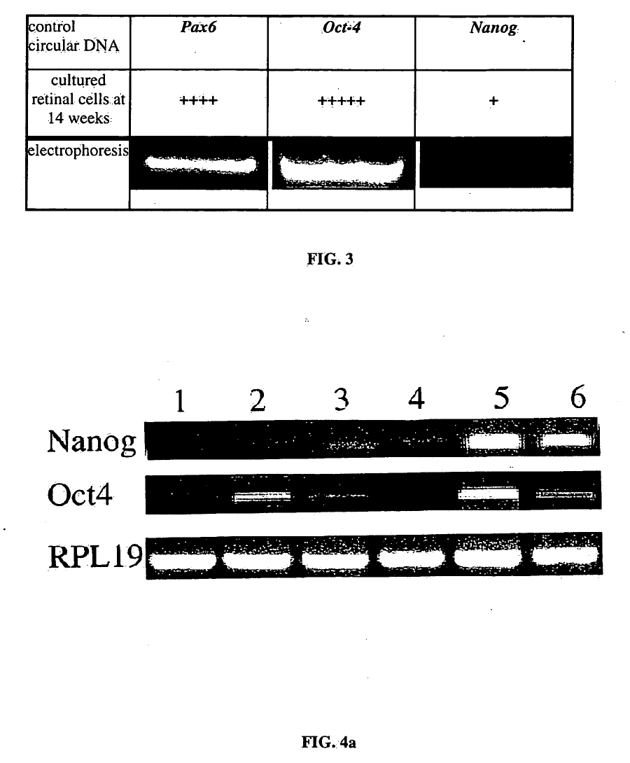

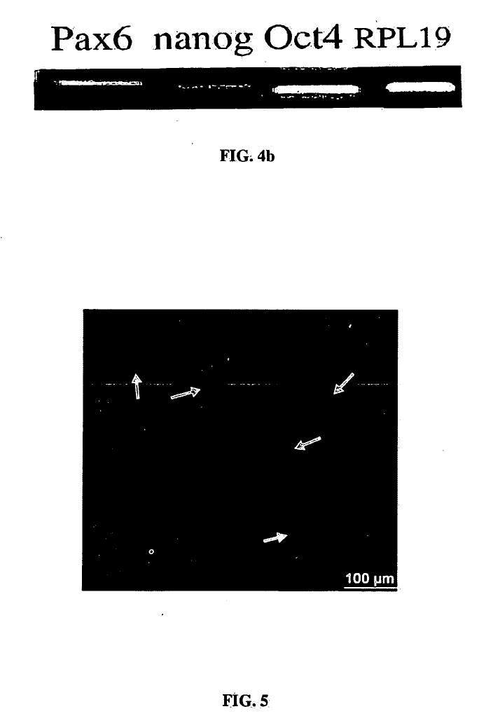

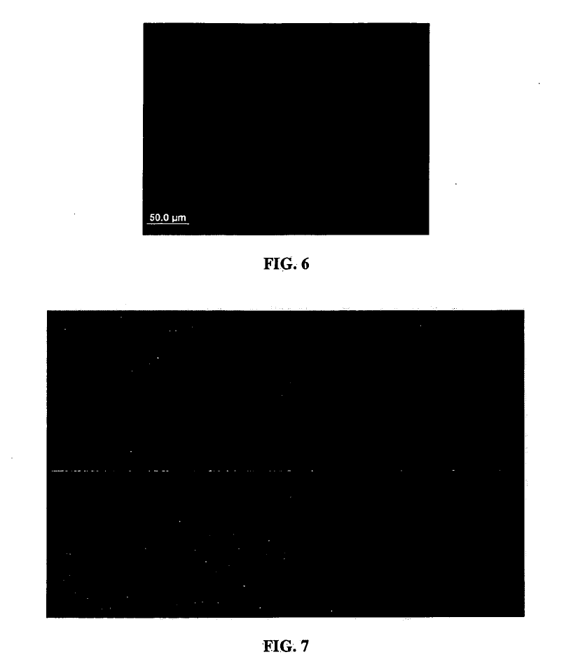

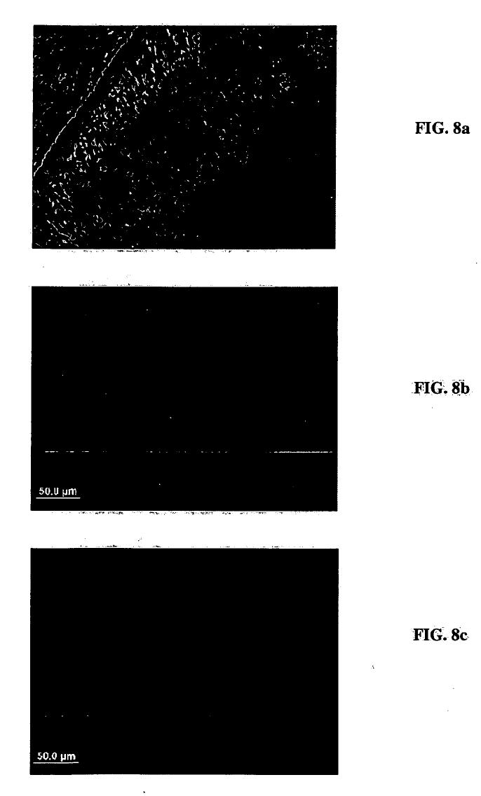

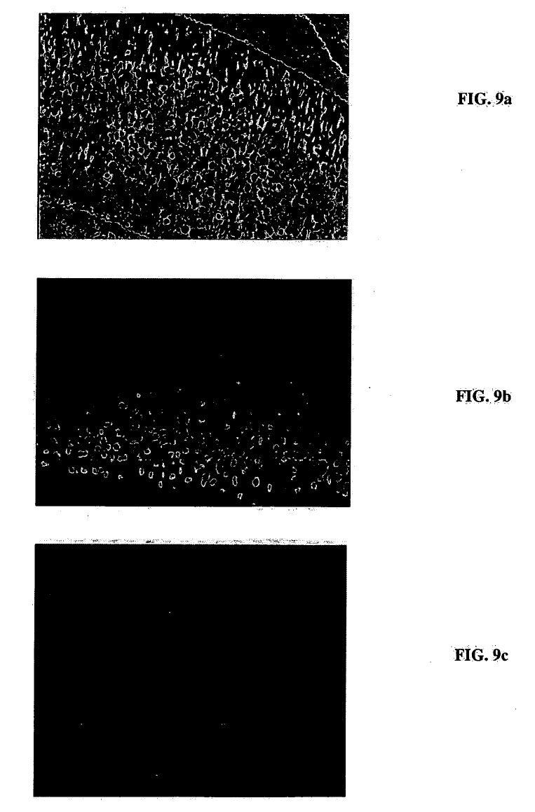

### DEFINITIONS

“Stem cell” as used herein refers to an undifferentiated cell haying the ability to both to self-renew and differentiate to produce at least one functional, terminal cell type.

“Prescursor cell” and “progenitor cell” are used interchangeable herein to refer to a lineage-committed cell capable of dividing and differentiating to form a particular terminal cell type. A neural precursor cell is a non-limiting example of a progenitor cell.

“Pluripotent” as used herein refers to the ability of a stem cell to differentiate into at least one cell type from each of the three embryonic germ layer lineages (i.e the ectoderm, endoderm and mesoderm).

“Multipotent” as used herein refers to refers to the ability of a stem cell to differentiate into various cells belonging to one embryonic germ layer lineage (i.e the ectoderm, endoderm or mesoderm).

“Embryonic-like” as used herein refers to the presence of characteristics that are demonstrated by embryonic stem cells. These characteristics include pluripotency and the presence of one or more markers selected from the group consisting of Nanog, OCT-4, SSEA-1, SSEA -3, SSEA-4, SOX-2, FGF-4, REX-1, and combinations thereof.

“Fetal” and “prenatal” are used interchangeably herein to refer to the period that precedes the birth of a fetus, beginning with the formation of a diploid zygote. Thus, in the context of the invention, tissues and their associated cells that are derived from a fetus prior to natural birth, or birth by cesarean section, are fetal (i.e. prenatal) tissues. Tissues obtained from mammalian tissue following the birth (e.g. live and still birth) of the mammal are adult tissues and cells derived therefrom are “adult cells.” Fetal tissue and fetal cells may be obtained from, for example, miscarried and aborted fetuses. The stem cells of the invention may be derived from fetal tissues, adult tissues, and combinations thereof.

The term “clone,” or “clonal cell,” refers to a single cell which is expanded to produce an isolated population of daughter cells (i.e. a “clonal cell population”).

The term “cell line” refers to one or more generations of cells which are derived from a clonal cell.

When used to refer to a cell, “derived from,” indicates that the cell came from a specific source such as for example, a tissue, a clonal cell line, a body fluid, or a primary cell culture. For example, when a cell population is expanded from a clonal cell that was isolated from the ectoderm germ layer, the cell population may be described as being derived from both the ectoderm germ layer and the clonal cell.

“Test agent” refers to any substance that is evaluated for its effect on one or more cell properties. For example, test agents may be evaluated for their effect on cell proliferation, cell differentiation, or cell death (i.e. toxicity). Test agents may also be evaluated for their ability to, cure, mitigate, treat, or prevent disease in a subject. Test/agents include, but are not limited to, chemical compounds, biologic agents, proteins, glycoproteins, peptides, nucleic acids, lipids, polysaccharides, supplements, signals, diagnostic agents and immune modulators. Test agents also include electromagenetic energy and mechanical forces.

The term “isolated,” or “purifed,” is used to describe a homogenous, or essentially homogenous, population of cells that has been separated from its natural environment. “Essentially homogenous” means that the cell population is about 30%, 35%, 40%, 45%, 50%, 55%, 60%, 65%, 70%, 75%, 80%, 85%, 90%, 95% or 99% free from other undesired cells, molecules and/or debris.

The phrases “the invention,” “the cells of the invention,” and “retinal stem cells” refer to stem cells that are derived from retinal tissue. Stem cells derived from the retinal pigment epithelium (“RPE stem cells”) are a non-limiting example of a retinal stem cell. Retinal stem cells may be derived from human, non-human sources, and combinations thereof. Sources for deriving the RPE stem cells of the invention include adult RPE tissues, fetal RPE tissues, and combinations thereof.

### DETAILED DESCRIPTION

The present inventors have discovered a unique population of stem cells residing in the retina. These cells have embryonic-like properties and are useful in a range of applications including regenerative medicine and bioanalytical research.

The stem cells of the invention may be found in the retina, in particular, the retinal pigment epithelium. The retina contains seven layers of alternating cells and processes that convert a light signal into a neural signal. The retinal pigment epithelium (RPE) is a layer of pigmented cuboidal cells that lies between the rod and cone receptors of the neurosensory retina and the underlying choroid. [Cassin, B. and Solomon, S. (2001). Dictionary of eye terminology. Gainesville, Fla. Triad Pub. Co. ISBN 0-937404-63-2].

The stem cells of the invention are characterized by their embryonic-like properties. That is, the stem cells of the invention (e.g. RPE stem cells), have a number of pluripotent cell characteristics such as, for example, the expression of embryonic stem cell markers. The stem cells of the invention express, among other markers, Nanog, OCT-4, SSEA-1, SSEA-3, SSEA-4, SOX-2, FGF-4, REX-1 and combinations thereof.

In one aspect of the invention, the retinal stem cells are derived from the retinal pigment epithelium. These cells are referred to as “RPE stem cells,” or “RPE cells.” RPE stem cells may be derived from any source of retinal pigment epithelium that provides a population of cells that expresses at least one of Nanog, OCT-4, SSEA-1, SSEA-3, SSEA-4, SOX-2, FGF-4, and REX-1. Suitable sources of retinal pigment epithelium for deriving RPE stem cells include adult retinal tissue, fetal retinal tissue, and combinations thereof.

In general, the RPE stem cells of the invention may be produced by obtaining retinal pigment epithelium tissue (“RPE tissue) from a donor source, disaggregating the RPE tissue, culturing the cells obtained from the disaggregated tissue, and selecting a cell or cell colony which expresses at least one of the markers selected from Nanog, OCT-4, SSEA-1, SSEA-3, SSEA-4, SOX-2, FGF-4, REX-1, and combinations thereof.

Harvesting of RPE tissue may accomplished by obtaining a donor eye and pinning it by the optic nerve stump into an eye cage and placing it in an eye jar immediately after corneal removal. The jar is then flooded to capacity with cold Delbecco's modified essential medium (DMEM) supplemented with 5% fetal bovine serum. After loose connective tissue and muscle have been carefully trimmed from the eye, it is placed upright on a sterile plate and the anterior segment with the adherent vitreous is lifted out of the eye cup. The neural retina is separated at the optic disc and removed. The shell is then washed with Hanks' balanced salt solution, Ca++ and Mg++ free, and treated with 0.25% trypsin for 30 min. at 37° C. The trypsin solution is aspirated from the shell and DMEM (GEBCO) is added. The RPE cells are released from Bruch's membrane by gently pipetting the culture medium in the shell. Details for the preparation of RPE tissue are presented in the following references, the disclosures of which are incorporated herein by reference: Pfeffer, B. A., Chapter 10, “Improved Methodology for Cell Culture of Human and Monkey Retinal Pigment Epithelium”, Progress in Retinal Research, Vol. 10 (1991); Ed. by Osborn, N. and Chader, J., or Mayerson, P. L., et al., “An Improved Method for Isolation and Culture of Rat Retinal Pigment Epithelial Cells,” Investigative Opththalmology & Visual Science, November, 1985, 26: 1599-1609; U.S. Pat. No. 6,117,675; U.S. Pat. No. 6,045,791; and Lane, C., et al. in Eye (1989) 3, 27-32.

RPE tissues for deriving the cells of the invention may be prepared by removing anterior portions of a donor eye (including the lenticular and corneal tissue) and making an incision through the sclera to facilitate the removal of the vitreous, retina and associated vasculature. The retinal cups, with exposed RPE, may then be enzymatically digested (i.e. in dispase) and subsequently digested in a high Mg.sup.2+, low Ca. sup.2+ aCSF solution for 10 min in the presence of trypsin (1.3 mg/mL), hyaluronidase (0.66 mg/mL), and kyneurinic acid (0.1 mg/mL). In the case of embryonic tissue, the retinal cups may be taken from dispase and separated from the basement membrane. The RPE can then be separated from its basement membrane (and associated choroidal tissue and vasculature).

Mechanical separation may be used to prepare RPE tissue with or without enzymatic digestion. Mechanical devices for this purpose include grinders, blenders, sieves, homogenizers, pressure, cells, or insonators (Freshney, Culture of Animal Cells. A Manual of Basic Technique, 2d Ed., A.R. Liss, Inc., New York, 1987, Ch. 9, pp. 107-26; incorporated herein by reference).

After disaggregating the RPE tissue to form a suspension of RPE cells, the RPE cells are grown in culture for clonal selection. Suitable media and culture conditions for growing RPE cells are available in the art as demonstrated by the following references, the disclosures of which are incorporated by reference: Pfeffer, B. A., Chapter 10, “Improved Methodology for Cell Culture of Human and Monkey Retinal Pigment Epithelium”, Progress in Retinal Research, Vol. 10 (1991); Ed. by Osborn, N. and Chader, J., or Mayerson, P. L., et al., “An Improved Method for Isolation and Culture of Rat Retinal Pigment Epithelial Cells”, Investigative Opththalmology & Visual Science, November, 1985, 26: 1599-1609. While in culture, single cells (or cell colonies of cells) demonstrating proliferation are isolated and subjected to further expansion as homogenous populations of clonal cells. These cell populations are then individually assayed for the presence of at least one of Nanog and OCT-4. Cell populations that show the presence of one or both of these markers are then selected for serial expansion and cryopreservation as an established cell line.

Assaying homogenous cultures of RPE cells for the presence of the desired markers (e.g. Nanog and OCT-4) may be accomplished through various means, including, but not limited to, oligonucleotide hybridization and immunohistochemical staining. Methods for the detection of these, and other markers, are detailed in the following references, the disclosures of which are incorporated herein by reference: Seigel et al. Mol. Vis. 2007 Jun. 8; 13:823-32; Hart et al. Dev Dyn. 2004 May; 230(1): 187-98; and Atlasi et al. Int J Cancer. 2007 Apr. 1; 120(7): 1598-602).

In one aspect of the invention, the retinal stem cells (e.g. RPE stem cells) of the invention express one or both of the markers OCT-4 and Nanog. Without being confined to any particular theory, the inventors note that these markers are only active in fully pluripotent cells capable of differentiating into each of the three germ layers (ectoderm, endoderm and mesoderm). Nanog and OCT-4 serve as transcription factors that maintain the pluripotency and self-renewal of embryonic stem cells (ESCs).

In some aspects of the invention, retinal stem cells (e.g. RPE stem cells) are used to prepare a pharmaceutical composition for use in therapeutic applications. Such compositions are made by suspending an appropriate amount of retinal stem cells in a pharmaceutically acceptable carrier. As used herein the phrase “pharmaceutically acceptable” means a carrier, or vehicle, that does not cause an adverse reaction when administered to a mammal. Such carriers are non-toxic and do not create an inflammatory or anergic response in the body. Pharmaceutically acceptable carriers for practicing the invention include any of the well known components useful for immunization such as, for example, culture media and phosphate buffered saline. Additional physiologically acceptable carriers and their formulations are well-known and generally described in, for example, Remington's Pharmaceutical Science (18.sup.th Ed., ed. Gennaro, Mack Publishing Co., Easton, Pa., 1990) and the Handbook of Pharmaceutical Excipients (4.sup.th ed., Ed. Rowe et al. Pharmaceutical Press, Washington, D.C.), each of which is incorporated by reference.

In some aspects of the invention, retinal stem cells (e.g. RPE stem cells) are used in an in vitro assay for evaluating the biological activity of a test agent. Test agents can be evaluated for a number of activities including cytotoxicity, cell proliferation, cell differentiation and therapeutic drag potential. In general, such in vitro assays involve obtaining a retinal stem cell, expanding the retinal stem cell to produce a population of retinal stem cells, exposing the population of retinal stem cells to a test agent, and determining the effect that the test agent has on the population of retinal stem cells.

The retinal stem cells of the invention can be used to measure test agent cytotoxicity. This may be accomplished by exposing a population of retinal stem cells to the test agent and measuring, for example, cell viability or cell proliferation. Assays for measuring cell viability are known in the art, and are described, for example, by the following references, the disclosure of which are incorporated by reference: Crouch et al. J. Immunol. Meth. 160, 81-8; Kangas et al. Med. Biol. 62, 338-43, 1984; Lundin et al., Meth. Enzymol. 133, 27-42, 1986; Petty et al. Comparison of J. Biolum. Chemilum. 10, 29-34, 1995; and Cree et al. Anticancer Drugs 6: 398-404, 1995.

In some aspects of the invention, retinal stem cells are used to determine the ability of a test agent to induce cell differentiation. The ability of a test agent to influence the differentiation of retinal stem cells may be determined by, for example, measuring changes in cell morphology and alkaline phosphatase (AP) activity. Details for measuring cell differentiation are disclosed in the following references, the disclosures of which are incorporated by reference: Wobus et al., Exp. Cell. Res. 152:212-219, 1984; and Pease et al., Dev. Bio. 141:344-352, 1990.

### EXAMPLE 1

### Solutions

CHFE solution

| | | |
|---|---|---|
| KCL | 5.4 | mmol/L |
| KH PO | 0.4 | mmol/L |
| MgSO | 0.8 | mmol/L |
| NaCl | 127 | mmol/L |
| Na HPO | 0.34 | mmol/L |
| EDTA | 2 | mmol/L |
| D-glucose | | |
| Phenol red (only part in Russian) | | |
| pH 7.2-7.4 | | |

### Enzyme Soln.

Solution collegenase 64 units/mg
Hyaluronidase 220 units/mg
CHFE (in Hank's EDTA soln.)

### EXAMPLE 2

### Isolation of Retinal Pigmented Epithelium Stem Cells

1. Collected human eyes were separated from separated from surrounding tissues using surgical microscope.
2. Eyes were washed in 70% ethanol
3. Eyes were placed in Hank's EDTA solution and washed 4×
4. After washing, the front chamber of eye was separated from the back eye chamber
5. In the area of optic nerve, the retina was cut and removed together with the vitreal body (if retina separation is not observed, then this step is skipped).
6. Enzyme solution was added to the back chamber of the eye and left for 8 min in thermostat@ 37 degrees
7. Back eye chamber was washed of enzymes and the retina was removed (if necessary) and then Hank's EDTA cold solution was added to the dish and left for 15 to 30 minutes
8. If separation of PE did not take place by the whole layer, pieces of PE were collected by pipette and all liquid with the cells was collected from the dish. If PE disassociated in the enzyme solution then this solution with cells was diluted 20-50× with Hank's EDTA solution. The obtained solution was centrifuged at 800 rpm and the supernatant was removed
9. To disassociate PE, the following procedures were used:
   a. for well digested PE preparations, pipetting for short time (mechanically)
   b. for less digested PE preparations, addition of 0.125% trypsin for 3 min., or 10 min. processing or treatment with agutase and short pipetting (if not sufficiently digested)

### EXAMPLE 3

### Cell Culture of RPE Stem Cells in Serum-Free Media

Cells obtained from Example 2 were cultured in serum-free media NeuroCult with addition of the following:
NueroCult Proliferation Supplement (Stem Cell Technology Inc, USA)
20 ng/ml EGF (Sigma, USA)
20 ng/ml FGF-2 (Sigma, USA)
2 microgram/ml Heparin (Sigma, USA)
0.1 g/l penicillin/streptpmycin (Sigma, USA)

Cells were cultured in 24 microwell plates at a density of 5×10⁴ cells in 1.5 ml media at 37 degrees centigrade in 5% CO₂. Partial replacement of medium was performed every 1-2 days.

Obtained spheres of RPE cells of human eye were passed every 1-2 weeks by using accutase for 10 minutes at 37 degrees centigrade. Cells were/washed of enzymes 3 times using cell culture medium.

### EXAMPLE 4

### Cell Culture of RPE Stem Cells in Serum-Supplemented Media

RPE cells were cultured in the following serum-containing medium:
DMEM/F12 with the addition of the following:
2% N2 (Sigma, USA)
20 ng/ml EGF (Sigma, USA)
20 ng/ml FGF-2 (Sigma, USA)
2 microgram/ml Heparin (Sigma, USA)
2 mM L-Glutamine (Sigma, USA)
0.1 g/l penicillin/streptomycin (Sigma, USA)
1% fetal bovine serum (FBS)
   Cells were cultured in 24 microwell plates at a density of 5×10⁴ cells in 1.5 ml media at 37 degrees centigrade in 5% CO₂. Partial replacement of medium was performed every 1-2 days.

After obtaining a cell monolayer, cells were passed using accutase for 1-2 min. Inhibition of accutase enzyme was done using serum.

### EXAMPLE 5

### PCR Analysis of RPE Stem Cells

Total RNA was isolated from the indicated eye tissues (TRI® Reagent, “Sigma”). mRNA was prepared using kit (Dynal Biotec ASA, Norway). mRNA was used for cDNA synthesis with reverse transcriptase (Superscript II; Invitrogen, USA) and oligo(dT)-primer or random hexanucleotide primers. The amount of synthesized cDNA was evaluated by PCR using primers specific for ribosomal protein Rpl19 depending on the source of RNA. PCR was performed with primers, constructed by mRNA nucleotide sequences of Nanog and Oct4 (NCBI BLAST).

Primers for PCR (pcr) and sequencing (sq).

| | | |
|---|---|---|
| No | Name | Nucleotide sequence |

| | | |
|---|---|---|
| 1. | pcrPax6dir. | 5′gtcatcaataaacagagttcttc3′ |
| 2. | pcrPax6rev. | 5′cgattagaaaaccatacctgtat3′ |
| 3. | pcrOCT-4 dir. | 5′atgtgtaagctgcggcccttg 3′ |
| 4. | pcrOCT-4 rev. | 5′gtttgaatgcatgggagagcc 3′ |
| 5. | sqOCT-4 dir. | 5′cgaccatctgccgctttgag3′ |
| 6. | sqOCT-4 rev. | 5′ccccctgtcccccattccta3′ |
| 7. | pcrNanogdir. | 5′gtgtggaiccagcttgtccc 3′ |
| 8. | pcrNanogrev. | 5′ctgcgtcacaccattgctattc 3′ |
| 9. | sqNanogdir. | 5′cctcctcttcctctatactaa3′ |
| 10. | sqNanogrev. | 5′ctgcgtcacaccattgctattc 3′ |
| 11. | RPL19dir | 5′agggtacagccaatgcccga 3′ |
| 12. | RPL19rev. | 5′ccttggataaagtcttgatgatc 3′ |

The PCR parameters were as follows: 3 minute at 94° followed by 30-40 cycles of 1 minute at 94°, 1 minute at 56°, 1 minute at 72° and final incubation for 5 minutes at 72° (Eppendorf amplifier).

PCR reaction products were analyzed by 1% agarose gel-electrophoresis on transilluminator BIO-RAD™XR. Primers for each gene were designed crossing introns, when possible.

Nucleotide sequence was analyzed by DNA sequenatbr (ABI. Prizm 3100, USA) using kit BigDye 1.1, USA.

### PCR Parameters (Eppendorf Amplifier):

3 min—94°; 1 min at 94°, 1 min at 56°, 1 min at 72°-30 cycles for RPL, 40 for Oct4 and Nanog; final incubation—5 min at 72°. PCR was performed with cDNA, synthesized with random hexanucleotide primers on mRNA (Dynal Biotec ASA, Norway) from RPE of adult eye (male, age 45, myocardial infarction). We have isolated total RNA using TRI® Reagent, “Sigma”.

We claim:

1. A composition comprising purified retinal pigment epithelial stem cells, wherein said stem cells express at least one of OCT-4 and Nanog.

2. The composition of claim 1, wherein said stem cells are derived from a tissue source selected from the group consisting of adult, fetal, and combinations thereof.

3. The composition of claim 1, wherein said stem cells are derived from a source selected from the group consisting of mammal, fish, bird, insect, and combinations thereof.

4. The composition of claim 3, wherein said mammal comprises a source selected from the group consisting of human, dog, cat, horse, and combinations thereof.

5. The composition of claim 1, wherein said stem cells are human stem cells.

6. The composition of claim 5, wherein said human stem cells are derived from a clone.

7. The composition of claim 1, wherein at least a portion of said stem cells are pluripotent cells.

8. The composition of claim 1, wherein said stem cells are multipotent cells.

9. The composition of claim 1, wherein said stem cells are precursor cells.

10. The composition of claim 8, wherein said stem cells are neural stem cells capable of forming at least one of a neuronal cell and a glial cell.

11. The composition of claim 10, wherein said glial cell is selected from the group consisting of an astroglial cell and an oligoglial cell.

12. A method for testing the differentiation capacity of a test agent comprising:
providing a composition comprising isolated pigment retinal epithelial stem cells wherein said stem cells expresses at least one of Nanog and OCT-4;
introducing to said composition said test agent; and
determining the ability of said test agent to affect the differentiation of said stem cells.

13. The composition of claim 12, wherein said stem cells are derived from a tissue source selected from the group consisting of adult, fetal, and combinations thereof.

14. The composition of claim 12, wherein said stem cells are derived from a source selected from the group consisting of mammal, fish, bird, insect, and combinations thereof.

15. The composition of claim 14, wherein said mammal comprises a source selected from the group consisting of human, dog, cat, horse, and combinations thereof.

16. The composition of claim 12, wherein said stem cells are human stem cells.

17. The composition of claim 16, wherein said human stem cells are derived from a clone.

18. The composition of claim 12, wherein at least a portion of said stem cells are pluripotent cells.

19. The composition of claim 12, wherein said stem cells are multipotent cells.

20. The composition of claim 12, wherein said stem cells are precursor cells.

21. The composition of claim 19, wherein said stem cells are neural stem cells capable of forming at least one of a neuronal cell and a glial cell.

22. The composition of claim 21, wherein said glial cell is selected from the group consisting of an astroglial cell and an oligoglial cell.


SEQUENCE LISTING (12 sequences)

SEQ ID NO: 1 - DNA, 23 bp, homo sapiens
gtcatcaataaacagagttcttc

SEQ ID NO: 2 - DNA, 23 bp, homo sapiens
cgattagaaaaccatacctgtat

SEQ ID NO: 3 - DNA, 21 bp, homo sapiens
atgtgtaagctgcggcccttg

SEQ ID NO: 4 - DNA, 21 bp, homo sapiens
gtttgaatgcatgggagagcc

SEQ ID NO: 5 - DNA, 20 bp, homo sapiens
cgaccatctgccgctttgag

SEQ ID NO: 6 - DNA, 20 bp, homo sapiens
ccccctgtcccccattccta

SEQ ID NO: 7 - DNA, 20 bp, homo sapiens
gtgtggatccagcttgtccc

SEQ ID NO: 8 - DNA, 22 bp, homo sapiens
ctgcgtcacaccattgctattc

SEQ ID NO: 9 - DNA, 21 bp, homo sapiens
cctcctcttcctctatactaa

SEQ ID NO: 10 - DNA, 22 bp, homo sapiens
ctgcgtcacaccattgctattc

SEQ ID NO: 11 - DNA, 20 bp, homo sapiens
agggtacagccaatgcccga

SEQ ID NO: 12 - DNA, 23 bp, homo sapiens
ccttggataaagtcttgatgatc